(19) United States
(12) Patent Application Publication
(10) Pub. No.: US 2024/0065959 A1
(43) Pub. Date: Feb. 29, 2024

(54) ODOR ELIMINATING COMPOSITION WITH MORPHOLINIUM AND ALGINATE COMPOUNDS

(71) Applicant: Blessing Solutions LLC, Cicero, IN (US)

(72) Inventors: Raymond E. Guerry, Brick, NJ (US); Ralph Blessing, Cicero, IN (US)

(21) Appl. No.: 18/240,528

(22) Filed: Aug. 31, 2023

Related U.S. Application Data
(60) Provisional application No. 63/402,737, filed on Aug. 31, 2022.

(51) Int. Cl. A61K 8/49; A61Q 5/02; A61Q 15/00; A61Q 19/10

(52) U.S. Cl. CPC A61K 8/49; A61Q 5/02; A61Q 15/00; A61Q 19/10; A61K 2800/596; A61K 2800/70; A61K 2800/80

(57) ABSTRACT

The present disclosure related generally to an odor reducing composition comprising a morpholinium compound present in an amount in the range of 0.1-1.5 wt. %; sodium alginate present in an amount in the range of 0.01-0.5 wt. %; a surfactant composition present in an amount in the range of 15-35 wt. %; and water present in an amount in the range of 50-90 wt. %.

### CROSS-REFERENCE TO RELATED APPLICATIONS

This application claims the benefit of U.S. Provisional Patent Application No. 63/402,737, filed on Aug. 31, 2022, which is hereby incorporated by reference in its entirety.

### FIELD

The present disclosure relates generally to compositions and methods for reducing odor, specifically for neutralizing malodor adhering to skin or hair.

### BACKGROUND

Malodor may be caused by a variety of factors. For example, malodor causing agents and everyday odors may adhere to the skin, hair, and/or body from exposure to the environment (e.g., mildew, smoke, or sewage) and some working conditions (e.g., chemicals). Personal hygiene and other lifestyle factors may also lead to malodor.

In particular, for those in the medical field, malodor is often caused by exposure to formaldehyde or other chemicals and is very difficult to counteract. For example, medical school and laboratory employees may be exposed to embalming fluid, which typically contains a mixture of formaldehyde, glutaraldehyde, methanol, and other solvents. Many medical school and laboratory employees complain that even with wearing one or two pairs of gloves, their hands can smell of formaldehyde or other chemicals afterward which can make food taste worse and affect sleep and social life.

Accordingly, there is a need in the art for improved compositions that can reduce odors, and in particular, can reduce malodors adhered to the skin, hair, and/or body.

### BRIEF SUMMARY

In one aspect, the present disclosure provides an odor reducing composition comprising:
a morpholinium compound present in an amount in the range of 0.1-1.5 wt. %;
sodium alginate present in an amount in the range of 0.01-0.5 wt. %;
a surfactant composition present in an amount in the range of 15-35 wt. %; and
water present in an amount in the range of 50-90 wt. %.

In another aspect, the present disclosure provides an odor reducing composition comprising:
soyaethyl morpholinium ethosulfate present in an amount in the range of 0.7-0.8 wt. %;
sodium alginate present in an amount in the range of 0.03-0.05 wt. %;
two or more surfactants present in an amount in the range of 15-25 wt. %; and
water present in an amount in the range of 60-80 wt. %.

In another aspect, the present disclosure provides a method for reducing malodor on skin or hair, the method comprising providing an odor reducing composition as described herein and applying the composition to the skin or hair.

In another aspect, the present disclosure provides an odor reducing product comprising an absorbent substrate and an odor reducing composition as described herein disposed on the absorbent substrate.

In another aspect, the present disclosure provides a method for reducing malodor on skin or hair, the method comprising providing an odor reducing product as described herein and applying the product to the skin or hair.

In another aspect, the present disclosure provides for a container comprising an odor reducing product as described herein.

### BRIEF DESCRIPTION OF FIGURES

The accompanying drawings are included to provide a further understanding of the compositions, products, and methods of the disclosure, and are incorporated in and constitute a part of this specification. The drawings illustrate one or more embodiment(s) of the disclosure, and together with the description serve to explain the principles and operation of the disclosure.

FIGS. 1A and 1B are pictures of an embodiment of the odor reducing product of the disclosure as described herein.

FIGS. 2A and 2B are pictures of an embodiment of the odor reducing product of the disclosure as described herein.

FIG. 3 is a graph of the odor reduction of a composition of the disclosure as described herein.

### DETAILED DESCRIPTION

Compositions, products, and methods for reducing odor are provided. As described above, malodor can be caused from a variety of factors. Counteracting malodor can be particularly difficult, especially when such odor adheres to the skin or hair. The present inventors have found a composition that reduces odors, and particularly those malodors adhered to skin and hair, which includes both a morpholinium compound and sodium alginate. Such a composition demonstrates improved odor reducing capabilities. In one embodiment, the odor reducing composition comprises a morpholinium compound present in an amount in the range of 0.1-1.5 wt. %; sodium alginate present in an amount in the range of 0.01-0.5 wt. %; a surfactant composition present in an amount in the range of 15-35 wt. %; and water present in an amount in the range of 50-90 wt. %.

In various embodiments of the present disclosure as described herein, the morpholinium compound is N-substituted. The morpholinium compound may be N-substituted with a group individually selected from C₁-C₂₄ alkyl, C₂-C₂₄ alkenyl, C₁-C₂₄ alkoxy, C₁-C₂₄ carboxylic acid, C₁-C₂₄ alcohol, or C₁-C₂₄ amine groups. The term “alkyl” as used herein, means a straight or branched chain hydrocarbon. The term “alkenyl” as used herein, means a straight or branched chain hydrocarbon and containing at least one carbon-carbon double bond. The term “alkoxy” as used herein, means an alkyl group, as defined herein, appended to the parent molecular moiety through an oxygen atom.

In some embodiments, the morpholinium compound is N-substituted with a group individually selected from C₂-C₂₄ (e.g., C₄-C₂₄, C₈-C₂₄, or C₁₂-C₂₄) alkenyl, C₂-C₂₀ (e.g., C₄-C₂₀, C₈-C₂₀, or C₁₂-C₂₀) alkenyl, or C₂-C₁₈ (e.g., C₄-C₁₈, C₈-C₁₈, or C₁₂-C₁₈) alkenyl groups. As described above, the term “alkenyl” as used herein, means a straight or branched chain hydrocarbon and containing at least one carbon-carbon double bond. For example, the C₂-C₂₄ (e.g., C₂-C₂₀ or C₂-C₁₈) alkenyl may be a C₂-C₂₄ (e.g., C₂-C₂₀ or C₂-C₁₈) monosaturated hydrocarbon or a C₄-C₂₄ (e.g., C₄-C₂₀ or C₄-C₁₈) polysaturated hydrocarbon.

In particular embodiments, the morpholinium compound is N-substituted with a C₁₈ alkenyl groups. For example, in some embodiments, the morpholinium compound is N-substituted with a group individually selected from a C₁₈ monosaturated hydrocarbon or a C₁₈ polysaturated hydrocarbon. For example, in some embodiments, the morpholinium compound is N-substituted with an oleic hydrocarbon and/or a linoleic hydrocarbon.

In some embodiments, the morpholinium compound is N-substituted with a group individually selected from C₁-C₂₄ (e.g., C₁-C₂₀, C₁-C₁₈, C₁-C₁₂, C₁-C₁₀, or C₁-C₅) alkyl groups. As described above, the term “alkyl” as used herein, means a straight or branched chain hydrocarbon. In various embodiments, the morpholinium compound is N-substituted with a group individually selected from methyl, ethyl, propyl, or butyl groups. For example, in some embodiments, the morpholinium compound is N-substituted with an ethyl group.

In various embodiments of the present disclosure as described herein, the morpholinium compound is N,N-disubstituted. For example, the morpholinium compound may be N,N-disubstituted with groups individually selected from C₁-C₂₄ alkyl, C₂-C₂₄ alkenyl, C₁-C₂₄ alkoxy, C₁-C₂₄ carboxylic acid, C₁-C₂₄ alcohol, or C₁-C₂₄ amine groups. In some embodiments, the morpholinium compound is N,N-disubstituted with groups individually selected from C₂-C₂₄ (e.g., C₄-C₂₄, C₈-C₂₄, or C₁₂-C₂₄) alkenyl groups and C₁-C₂₄ (e.g., C₁-C₂₀, C₁-C₁₈, C₁-C₁₂, C₁-C₁₀, or C₁-C₅) alkyl groups. In some embodiments, the morpholinium compound is N,N-disubstituted with groups individually selected from C₂-C₂₀ (e.g., C₄-C₂₀, C₈-C₂₀, or C₁₂-C₂₀) alkenyl groups and C₁-C₂₀ (e.g., C₁-C₁₈, C₁-C₁₂, C₁-C₁₀, or C₁-C₅) alkyl groups. In some embodiments, the morpholinium compound is N,N-disubstituted with groups individually selected from C₂-C₁₈ (e.g., C₄-C₁₈, C₈-C₁₈, or C₁₂-C₁₈) alkenyl groups and C₁-C₁₈ (e.g., C₁-C₁₂, C₁-C₁₀, or C₁-C₅) alkyl groups. For example, the C₂-C₂₄ (e.g., C₂-C₂₀ or C₂-C₁₈) alkenyl may be a C₂-C₂₄ (e.g., C₂-C₂₀ or C₂-C₁₈) monosaturated hydrocarbon or a C₄-C₂₄ (e.g., C₄-C₂₀ or C₄-C₁₈) polysaturated hydrocarbon.

In various embodiments, the morpholinium compound is N,N-disubstituted with groups individually selected from C₁₈ alkenyl, methyl, ethyl, propyl, and butyl groups. In some embodiments, the morpholinium compound is N,N-disubstituted with a C₁₈ alkenyl and an ethyl group. For example, in various embodiments, the morpholinium compound is N,N-disubstituted with an oleic hydrocarbon, a linoleic hydrocarbon, and/or an ethyl group. In some embodiments, the morpholinium compound is N,N-disubstituted with an oleic hydrocarbon and an ethyl group. In some embodiments, the morpholinium compound is N,N-disubstituted with a linoleic hydrocarbon and an ethyl group. In various embodiments, the morpholinium compound is monocyclic and not C-substituted.

In various embodiments, the morpholinium compound is a natural product. For example, in various embodiments, the morpholinium compound may be derived from plant sources (e.g., soy). In particular embodiments, the morpholinium compound is soyaethyl morpholinium ethosulfate.

The counterion of the morpholinium compound is not particularly limited. In some embodiments, the counterion is a sulfate, phosphate, nitrate, or acetate. In various embodiments, the counterion is an alkylsulfate. For example, in some embodiments of the disclosure as described herein, the counterion is a methosulfate, ethosulfate, propylsulfate, or a butylsulfate. In particular embodiments, the counterions is an ethosulfate.

As described above, the odor reducing composition includes a morpholinium compound present in an amount in the range of 0.1-1.5 wt. %. In some embodiments, the morpholinium compound is present in an amount in the range of 0.1-1.25 wt. %. For example, in various embodiments, the morpholinium compound is present in the range of 0.2-1.25 wt. %, or 0.3-1.25 wt. %, or 0.5-1.25 wt. %, or 0.7-1.25 wt. %. In some embodiments, the morpholinium compound is present in an amount in the range of 0.1-1 wt. %. For example, in various embodiments, the morpholinium compound is present in the range of 0.2-1 wt. %, or 0.3-1 wt. %, or 0.5-1 wt. %, or 0.7-1 wt. %. In some embodiments, the morpholinium compound is present in an amount in the range of 0.1-0.8 wt. %. For example, in various embodiments, the morpholinium compound is present in the range of 0.2-0.8 wt. %, or 0.3-0.8 wt. %, or 0.5-0.8 wt. %, or 0.7-0.8 wt. %. In particular embodiments, the morpholinium compound is present in an amount of 0.76 wt. %.

As described above, the odor reducing composition also includes sodium alginate, present in an amount in the range of 0.01-0.5 wt. %. In some embodiments of the present disclosure as described herein, the sodium alginate is present in the range of 0.01-0.2 wt. %. For example, in various embodiments, the sodium alginate compound is present in the range of 0.02-0.2 wt. % or 0.03-0.2 wt. %. In some embodiments, the sodium alginate is present in the range of 0.01-0.1 wt. %. For example, in various embodiments, the sodium alginate compound is present in the range of 0.02-0.1 wt. % or 0.03-0.1 wt. %. In some embodiments, the sodium alginate is present in the range of 0.01-0.05 wt. %. For example, in various embodiments, the sodium alginate compound is present in the range of 0.02-0.05 wt. % or 0.03-0.05 wt. %. In particular embodiments, the sodium alginate compound is present in an amount of 0.04 wt. %.

The present inventors have found that including both a morpholinium compound and sodium alginate in the composition as described herein provides advantageous odor reducing properties to the composition. In particular, the present inventors have found that inclusion of both the morpholinium compound and sodium alginate provide synergistic effects to the odor reducing properties of the composition. As such, both the morpholinium compound and the sodium alginate provide the advantageous properties of the composition. In some embodiments of the present disclosure as described herein, the weight ratio of the morpholinium compound to the sodium alginate is at least 5:1. For example, in various embodiments, the weight ratio of the morpholinium compound to the sodium alginate is at least 10:1, or 15:1, or 20:1. In some embodiments of the present disclosure as descried herein, the weight ratio of the morpholinium compound to the sodium alginate is in the range of 5:1 to 20:1 (e.g., 10:1 to 20:1, or 15:1 to 20:1).

As described above, the odor reducing composition also includes a surfactant composition. In various embodiments, the surfactant composition comprises one or more surfactants. In some embodiments, the one or more surfactants are cationic, anionic, or zwitterionic. In some embodiments, the one or more surfactants is an alkyl sulfate. For example, in some embodiments, the one or more surfactants is sodium laureth sulfate. In some embodiments, the one or more surfactants is a fatty acid amine. For example, in some embodiments, the one or more surfactants is cocamidopropyl betaine. In some embodiments of the disclosure as described herein, the one or more surfactants comprise both sodium laureth sulfate and cocamidopropyl betaine.

The one or more surfactants may be present in the surfactant composition in various amounts. For example, in some embodiment, the one or more surfactants are present in the surfactant composition in an amount in the range of 50-99 wt. %, or 50-95 wt. %, or 50-90 wt. %, or 50-85 wt. %, or 60-99 wt. %, or 60-95 wt. %, or 60-90 wt. %, or 60-85 wt. %, or 70-99 wt. %, or 70-95 wt. %, or 70-90 wt. %, or 70-85 wt. %, or 75-99 wt. %, or 75-95 wt. %, or 75-90 wt. %, or 75-85 wt. %.

The surfactant composition may further include one or more additional additives. Such additives are not particularly limited. For example, stabilizing agents, pH modifiers, chelating agents, viscosity modifiers, preservatives, antibacterial compounds, and/or alkaline salt compound may be included in the surfactant composition. In some embodiments, the surfactant composition further comprises one or more of ethanol, glycerin, lauramine oxide, EDTA (ethylenediaminetetraacetic add), and NaCl. In some embodiments, the surfactant composition further comprises ethanol, glycerin, lauramine oxide, EDTA (ethylenediaminetetraacetic acid), and NaCl. The amount of the additional additives is nor particularly limited. For example, the one or more additional additives may be present in the surfactant composition in an amount in the range of 5-40 wt. %, or 5-30 wt. %, or 5-20 wt. %, or 5-15 wt. %, or 10-40 wt. %, or 10-30 wt. %, or 10-20 wt. %, or 10-15 wt. %, or 15-40 wt. %, or 15-30 wt. %, or 15-20 wt. %.

As described above, water is also present in the odor reducing composition. Water may be present in the odor reducing composition is various amounts. For example, in various embodiments, water is present in an amount in the range of 55-90 wt. %, or 55-85 wt. %, or 55-80 wt. %, or 55-75 wt. %, or 60-90 wt. %, or 60-85 wt. %, or 60-80 wt. %, or 60-75 wt. %, or 65-90 wt. %, or 65-85 wt. %, or 65-80 wt. %, or 65-75 wt. %.

Another aspect of the present disclosure provides for an odor reducing composition comprising soyaethyl morpholinium ethosulfate present in an amount in the range of 0.7-0.8 wt. %; sodium alginate present in an amount in the range of 0.03-0.05 wt. %; two or more surfactants present in an amount in the range of 15-25 wt. %; and water present in an amount in the range of 60-80 wt. %. The two or more surfactants may be selected from the surfactants as described herein. For example, in some embodiments, the two or more surfactants comprise sodium laureth sulfate and cocamidopropyl betaine.

Like the surfactant composition, the odor reducing composition as described herein may further include one or more additional additives. For example, such additional additives may be selected from stabilizing agents, pH modifiers, fragrances, chelating agents, viscosity modifiers, preservatives, and antibacterial compounds. The additional additives may be present in the odor reducing composition in various amounts. For example, the one or more additional additives may be present in an amount in the range of 1-10 wt. %, or 1-8 wt. %, or 1-5 wt. %, or 2-10 wt. %, or 2-8 wt. %, or 2-5 wt. %.

In various embodiments, the odor reducing composition of the disclosure as described herein has a pH in the range of 5-8. For example, the odor reducing composition may have a pH in the range of 5.5-8, or 5.5-7.5, or 6-8, or 6-7.5, or 7-8, or 7-7.5.

The form of the odor reducing composition is not particularly limited. In various embodiments, the odor reducing composition is easily applicable to skin and hair (e.g., human skin or animal skin, or human hair or animal hair). For example, in some embodiments, the composition is in the form of a skin wash (e.g., a gel or foaming hand wash, or a gel or foaming body wash). In some embodiments, the odor reducing composition is in the form of a spray. In some embodiments, the odor reducing composition is in the form of a shampoo.

As described above, malodor may be caused by a variety of factors and can often linger and adhere to the skin and hair. As such, the odor reducing composition of the present disclosure is particularly useful in reducing malodor. Such malodor may be from a variety of sources and is not particularly limited. For example, the malodor may be from formaldehyde, mildew, sewage, body odor, or smoke.

Another aspect of the present disclosure as described herein provides a method for reducing malodor on skin or hair. The method includes providing an odor reducing composition as described herein and applying the odor reducing composition to the skin or hair.

Another aspect of the present disclosure as described herein provides an odor reducing product including an absorbent substrate and an odor reducing composition as described herein disposed on the absorbent substrate.

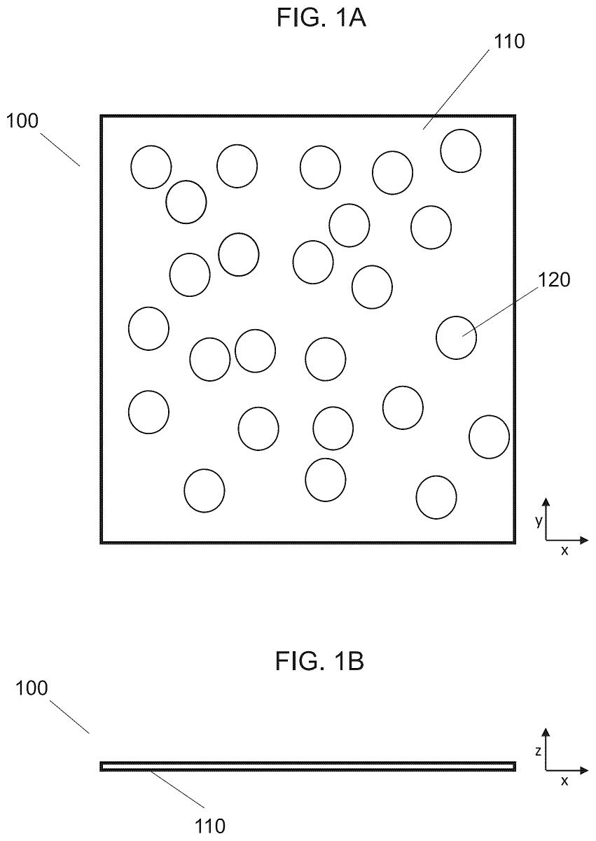

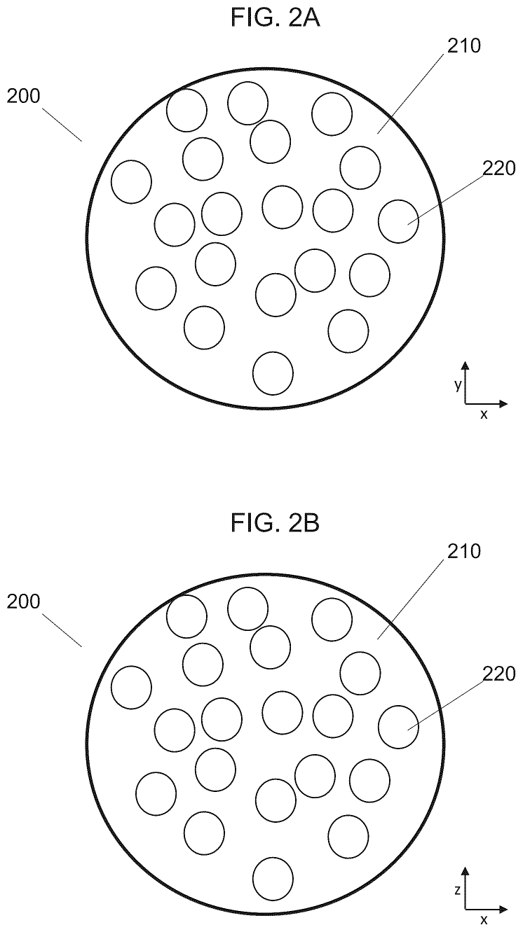

The shape of the absorbent substrate is not particularly limited. In some embodiments, the absorbent substrate is in a substantially flat form (e.g., a sheet, a pad, or a swab). Such an embodiment of the odor reducing product is shown in FIGS. 1A and 1B. The odor reducing product (100) includes an absorbent substrate (110) with the odor reducing composition (120) as described herein disposed on the absorbent substrate. FIG. 1B depicts the substantially flat form of the absorbent substrate (110) by providing a different perspective on the odor reducing product (100). In some embodiments, the absorbent substrate is in a substantially round form (e.g., a sphere, a ball, or a tube). Such an embodiment of the odor reducing product is shown in FIGS. 2A and 2B. The odor reducing product (200) includes an absorbent substrate (210) with the odor reducing composition (220) as described herein disposed on the absorbent substrate. FIG. 2B depicts the substantially round form of the absorbent substrate (210) by providing a different perspective on the odor reducing product (200).

While the odor reducing compositions (120, 220) in FIGS. 1A, 2A, and 2B are depicted as not covering the entire absorbent substrate (110, 210), such a depiction is not to be taken as limiting. The odor reducing composition may be disposed on the absorbent substrate in varying amounts and to varying degrees in either a heterogeneous or homogeneous manner. For example, the odor reducing composition may be disposed on at least 50 wt. % (e.g., at least 60 wt. %, 70 wt. %, 80 wt. %, or 90 wt. %) by surface area or volume of the absorbent substrate.

The absorbent substrate of the odor reducing product is not particularly limited and may be selected from any absorbing material (e.g., any substrate that can absorb liquids or gels). In some embodiments, the absorbent substrate is porous. In some embodiments, the absorbent substrate may be selected from any absorbing material. For example, in some embodiments, the absorbent substrate may be fabric (e.g., a woven or unwoven fabric), a foam, or a sponge. In various embodiments, the absorbent substrate comprises cellulose, polyester, or combinations thereof.

Another aspect of the present disclosure provides for a method for reducing malodor on skin or hair. The method includes providing an odor reducing product as described herein and applying the odor reducing product to the skin or the hair.

Another aspect of the present disclosure provides a container that includes the odor reducing product as described herein. The shape of the container is not particularly limited and may be selected to adequately fit at least one of the odor reducing products within.

### EXAMPLES

The Examples that follow are illustrative of specific embodiments of the compositions, products, and methods of the disclosure, and various uses thereof. They are set forth for explanatory purposes only, and are not to be taken as limiting the scope of the disclosure.

### Example 1. Preparation of an Odor Reducing Composition

An odor reducing composition, particularly useful as a hand wash was prepared by first preparing a surfactant composition. The components of the surfactant composition are recited in Table 1.

TABLE 1

| Ingredient | wt. % |
|---|---|
| Sodium Laureth Sulfate | 48.0 |
| Cocamidopropyl Betaine | 37.4 |
| Ethanol | 3.9 |
| Glycerin | 0.7 |
| Lauramine Oxide | 1.7 |
| EDTA | 4.4 |
| NaCl | 3.9 |
| Total | 100 |

The components listed in Table 1 were mixed together to provide the surfactant composition. The odor reducing composition was prepared by adding 25 wt. % of the surfactant composition to 70.82 wt. % water with 0.76 wt. % soya morpholinium ethosulfate, 0.04 wt. % sodium alginate, and other additives. The full formulation of the odor reducing composition is described in Table 2.

TABLE 2

| Ingredient | wt. % |
|---|---|
| Surfactant Composition | 25.00 |
| Soya Morpholinium Ethosulfate | 0.76 |
| Neutral Air | 0.11 |
| Aloe 200x | 0.04 |
| Glycerin | 1.00 |
| Xanthan Gum | 0.75 |
| Citric Acid | 0.34 |
| Sodium Alginate | 0.04 |
| EDTA | 0.08 |
| Phenoxyethanol | 0.50 |
| Fragrance | 0.57 |
| Water | 70.82 |
| Total | 100 |

### Example 2. Test of Odor Reduction

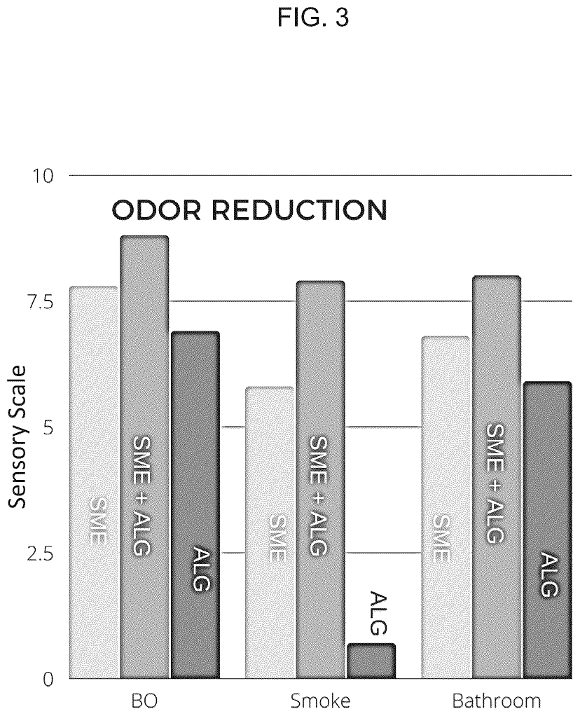

The odor reducing composition with both soya morpholinium ethosulfate and sodium alginate (SME+ALG), as described in Example 1, was then tested for its odor reducing properties. To do so, 1 gram of a concentrated malodor solution standard was sprayed onto a porous substrate. Then, 0.4 grams of a 10% solution of the odor reducing composition of Example 1 was sprayed onto the substrate and evaluated by a 3 person sensory panel. The 10% dilution of the odor reducing composition simulates a concentration that would be present during handwashing. The panel then ranked the malodor on a scale of 0-9 (9=very strong malodor, 5=moderate malodor, and 0=no malodor). As a comparison, two comparison composition were also prepared. The first comparative composition was made according to the same composition as reported in Table 2, but without sodium alginate and will be referred to as the SME (soya morpholinium ethosulfate) formulation. The second comparative composition was made according to the same composition as reported in Table 2, but without soya morpholinium ethosulfate and will be referred to as the ALG (sodium alginate) formulation. The results of the odor reducing tests against three malodors (BO, smoke, and bathroom) for the three formulations (SME, ALG, SME+ALG) are reported in FIG. 3.

From FIG. 3, it is clear that the SME+ALG formulation is the most effective for reducing odor for all malodors tested. Further still, the SME+ALG formulation shows more than just an additive improvement when compared to the SME formulation and ALG formulation. Without being bound by theory, the present inventors believe that the composition with both soya morpholinium ethosulfate and sodium alginate provide synergistic effects to the odor reducing properties of the formulation.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice. Thus, before the disclosed processes and devices are described, it is to be understood that the aspects described herein are not limited to specific embodiments, apparatuses, or configurations, and as such can, of course, vary. It is also to be understood that the terminology used herein is for the purpose of describing particular aspects only and, unless specifically defined herein, is not intended to be limiting.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. It will be further understood that the endpoints of each of the ranges are significant both in relation to the other endpoint, and independently of the other endpoint.

All methods described herein can be performed in any suitable order of steps unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

As will be understood by one of ordinary skill in the art, each embodiment disclosed herein can comprise, consist essentially of or consist of its particular stated element, step, ingredient or component. As used herein, the transition term “comprise” or “comprises” means includes, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment.

Unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Some embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

Additional aspects of the disclosure are provided by the following enumerated embodiments, which may be combined in any number and in any combination that is not logically or technically inconsistent.

Embodiment 1. An odor reducing composition comprising:
a morpholinium compound present in an amount in the range of 0.1-1.5 wt. %;
sodium alginate present in an amount in the range of 0.01-0.5 wt. %;
a surfactant composition present in an amount in the range of 15-35 wt. %; and
water present in an amount in the range of 50-90 wt. %.

Embodiment 2. The odor reducing composition of embodiment 1, wherein the morpholinium compound is N-substituted with a group selected from C₁₂-C₁₈ alkenyl groups.

Embodiment 3. The odor reducing composition of any of embodiment 1 or embodiment 2, wherein the morpholinium compound is N-substituted with a group selected from methyl, ethyl, propyl, or butyl groups.

Embodiment 4. The odor reducing composition of any of embodiments 1-3, wherein the morpholinium compound is N,N-disubstituted.

Embodiment 5. The odor reducing composition of embodiment 4, wherein the morpholinium compound is N,N-disubstituted with groups individually selected from C₁₂-C₁₈ alkenyl, methyl, ethyl, propyl and butyl groups.

Embodiment 6. The odor reducing composition of any of embodiments 1-5, wherein the morpholinium compound is soyaethyl morpholinium ethosulfate.

Embodiment 7. The odor reducing composition of any of embodiments 1-6, wherein the morpholinium compound is present in an amount in the range of 0.1-1.25 wt. % (e.g. in the range of 0.2-1.25 wt. %, or 0.3-1.25 wt. %, or 0.5-1.25 wt. %, or 0.7-1.25 wt. %, or 0.1-1 wt. %, or 0.2-1 wt. %, or 0.3-1 wt. %, or 0.5-1 wt. %, or 0.7-1 wt. %, or 0.1-0.8 wt. %, or 0.2-0.8 wt. %, or 0.3-0.8 wt. %, or 0.5-0.8 wt. %, or 0.7-0.8 wt. %).

Embodiment 8. The odor reducing composition of any of embodiments 1-7, wherein the sodium alginate is present in the range of 0.01-0.2 wt. % (e.g., in the range of 0.02-0.2 wt. %, or 0.03-0.2 wt. %, or 0.01-0.1 wt. %, or 0.02-0.1 wt. %, or 0.03-0.1 wt. %, or 0.01-0.05 wt. %, or 0.02-0.05 wt. %, or 0.03-0.05 wt. %).

Embodiment 9. The odor reducing composition of any of embodiments 1-8, wherein the weight ratio of the morpholinium compound to the sodium alginate is at least 5:1 (e.g., at least 10:1, or 15:1, or 20:1).

Embodiment 10. The odor reducing composition of any of embodiments 1-9, wherein the surfactant composition comprises one or more surfactants.

Embodiment 11. The odor reducing composition of embodiment 10, wherein the one or more surfactants is an alkyl sulfate (e.g., sodium laureth sulfate).

Embodiment 12. The odor reducing composition of embodiment 10, wherein the one or more surfactants is a fatty acid amine (e.g., cocamidopropyl betaine).

Embodiment 13. The odor reducing composition of any of embodiments 10-12, wherein the one or more surfactants are present in the surfactant composition in an amount in the range of 50-99 wt. % (e.g., in the range of 50-95 wt. %, or 50-90 wt. %, or 50-85 wt. %, or 60-99 wt. %, or 60-95 wt. %, or 60-90 wt. %, or 60-85 wt. %, or 70-99 wt. %, or 70-95 wt. %, or 70-90 wt. %, or 70-85 wt. %, or 75-99 wt. %, or 75-95 wt. %, or 75-90 wt. %, or 75-85 wt. %).

Embodiment 14. The odor reducing composition of any of embodiments 1-14, wherein the surfactant composition comprises one or more additional additives (e.g., stabilizing agent, pH modifier, chelating agent, viscosity modifier, preservatives, antibacterial compounds, alkaline salt compounds).

Embodiment 15. The odor reducing composition of embodiment 15, wherein the one or more additional additives are present in the surfactant composition in an amount in the range of 5-40 wt. % (e.g., in the range of 5-30 wt. %, or 5-20 wt. %, or 5-15 wt. %, or 10-40 wt. %, or 10-30 wt. %, or 10-20 wt. %, or 10-15 wt. %, or 15-40 wt. %, or 15-30 wt. %, or 15-20 wt. %).

Embodiment 16. The odor reducing composition of any of embodiments 1-16, wherein water is present in an amount in the range of 55-90 wt. % (e.g., 55-85 wt. %, or 55-80 wt. %, or 55-75 wt. %, or 60-90 wt. %, or 60-85 wt. %, or 60-80 wt. %, or 60-75 wt. %, or 65-90 wt. %, or 65-85 wt. %, or 65-80 wt. %, or 65-75 wt. %).

Embodiment 17. An odor reducing composition comprising:
soyaethyl morpholinium ethosulfate present in an amount in the range of 0.7-0.8 wt. %;
sodium alginate present in an amount in the range of 0.03-0.05 wt. %;
two or more surfactants present in an amount in the range of 15-25 wt. %; and
water present in an amount in the range of 60-80 wt. %.

Embodiment 18. The odor reducing composition of embodiment 18, wherein the two or more surfactants comprise sodium laureth sulfate and cocamidopropyl betaine.

Embodiment 19. The odor reducing composition of any of embodiments 1-18, further comprising one or more additional additives (e.g., stabilizing agent, pH modifier, fragrance, chelating agent, viscosity modifier, preservatives, antibacterial compounds).

Embodiment 20. The odor reducing composition of embodiment 19, wherein the one or more additional additives are present in an amount in the range of 1-10 wt. % (e.g., in the range of 1-8 wt. %, or 1-5 wt. %, or 2-10 wt. %, or 2-8 wt. %, or 2-5 wt. %).

Embodiment 21. The odor reducing composition of any of embodiments 1-20 having a pH in the range of 5-8 (e.g., in the range of 5.5-8, or 5.5-7.5, or 6-8, or 6-7.5, or 7-8, or 7-7.5).

Embodiment 22. The odor reducing composition of any of embodiments 1-21, wherein the composition is in the form of a skin wash (e.g., gel or foaming hand wash, or a gel or foaming body wash), a spray, or a shampoo.

Embodiment 23. A method for reducing malodor on skin or hair, the method comprising:
providing the odor reducing composition of any of embodiments 1-22; and
applying the odor reducing composition to the skin or hair.

Embodiment 24. An odor reducing product comprising:
an absorbent substrate; and
an odor reducing composition of any of embodiments 1-22 disposed on the absorbent substrate.

Embodiment 25. The odor reducing product of embodiment 24, wherein the absorbent substrate is porous.

Embodiment 26. The odor reducing product of embodiment 24 or embodiment 25, wherein the absorbent substrate is in a substantially flat form (e.g., a sheet, a pad, or a swab).

Embodiment 27. The odor reducing product of embodiment 24 or embodiment 25, wherein the absorbent substrate is in a substantially round form (e.g., a sphere, a ball, or a tube).

Embodiment 28. The odor reducing product of any of embodiments 24-27, wherein the absorbent substrate is a fabric (e.g., woven or unwoven), a foam, or a sponge.

Embodiment 29. The odor reducing product of any of embodiments 24-28, wherein the absorbent substrate comprises cellulose, polyester, or combinations thereof.

Embodiment 30. A method for reducing malodor on skin or hair, the method comprising:
providing the odor reducing product of any of embodiments 24-29; and
applying the odor reducing product to the skin or hair.

Embodiment 31. A container comprising an odor reducing product of any of embodiments 24-29.

We claim:

1. An odor reducing composition comprising:
a morpholinium compound present in an amount in the range of 0.1-1.5 wt. %;
sodium alginate present in an amount in the range of 0.01-0.5 wt. %;
a surfactant composition present in an amount in the range of 15-35 wt. %; and
water present in an amount in the range of 50-90 wt. %.

2. The odor reducing composition of claim 1, wherein the morpholinium compound is N,N-disubstituted.

3. The odor reducing composition of claim 1, wherein the morpholinium compound is N,N-disubstituted with groups individually selected from C₁₂-C₁₈ alkenyl, methyl, ethyl, propyl and butyl groups.

4. The odor reducing composition or claim 1, wherein the morpholinium compound is soyaethyl morpholinium ethosulfate.

5. The odor reducing composition of claim 1, wherein the morpholinium compound is present in an amount in the range of 0.1-1.25 wt. %.

6. The odor reducing composition of claim 1, wherein the sodium alginate is present in the range of 0.01-0.2 wt. %.

7. The odor reducing composition of claim 1, wherein the weight ratio of the morpholinium compound to the sodium alginate is at least 5:1.

8. The odor reducing composition of claim 1, wherein the surfactant composition comprises one or more surfactants.

9. The odor reducing composition of claim 8, wherein the one or more surfactants is an alkyl sulfate.

10. The odor reducing composition of claim 8, wherein the one or more surfactants is a fatty acid amine.

11. The odor reducing composition of claim 8, wherein the one or more surfactants are present in the surfactant composition in an amount in the range of 50-99 wt. %.

12. The odor reducing composition of claim 1, wherein the surfactant composition comprises one or more additional additives present in an amount in the range of 5-40 wt. %.

13. The odor reducing composition of claim 1, wherein water is present in an amount in the range of 55-90 wt. %.

14. The odor reducing composition of claim 1, further comprising one or more additional additives present in an amount in the range of 1-10 wt. %.

15. The odor reducing composition of claim 1 having a pH in the range of 5-8.

16. The odor reducing composition of claim 1, wherein the composition is in the form of a skin wash, a spray, or a shampoo.

17. A method for reducing malodor on skin or hair, the method comprising:
providing the odor reducing composition of claim 1; and
applying the odor reducing composition to the skin or hair.

18. An odor reducing product comprising:
an absorbent substrate; and
an odor reducing composition of claim 1 disposed on the absorbent substrate.

19. An odor reducing composition comprising:
soyaethyl morpholinium ethosulfate present in an amount in the range of 0.7-0.8 wt. %;
sodium alginate present in an amount in the range of 0.03-0.05 wt. %;
two or more surfactants present in an amount in the range of 15-25 wt. %; and
water present in an amount in the range of 60-80 wt. %.

20. The odor reducing composition of claim 19, wherein the two or more surfactants comprise sodium laureth sulfate and cocamidopropyl betaine.
